(19) United States
(12) Patent Application Publication
(10) Pub. No.: US 2026/0102626 A1
(43) Pub. Date: Apr. 16, 2026

(54) Physiotherapy Instrument

(71) Applicant: Dongguan Laiguang Electronic Technology Co., Ltd., Dongguan (CN)

(72) Inventors: Zirong WU, Dongguan (CN); Shunyang TANG, Dongguan (CN); Yong HUANG, Dongguan (CN); Liang WEN, Dongguan (CN)

(21) Appl. No.: 19/232,996

(22) Filed: Jun. 10, 2025

(30) Foreign Application Priority Data
Oct. 16, 2024 (CN) 2024224977633

(51) Int. Cl. A61N 5/06; A61N 5/00

(52) U.S. Cl. CPC A61N 5/06; A61N 2005/005; A61N 2005/063; A61N 2005/0644; A61N 2005/0665

(57) ABSTRACT

A physiotherapy instrument includes a housing, a light source and a heat-conducting base. The light source is provided inside the housing, and the light source outputs light and can generate heat during operation. The heat-conducting base is provided on the housing and supports the light source. The heat-conducting base is located between the housing and the light source. The heat-conducting base exchanges heat with the light source, transmits the heat generated by the light source to the housing, and dissipates the heat through the housing. At this time, the heat-conducting base is added relative to the light source, it is convenient for the heat generated by the light source during operation to be conducted to the housing through the heat-conducting base.

### CROSS-REFERENCE TO RELATED APPLICATIONS

The present application claims priority to a Chinese patent application No. 202422497763.3, titled "Physiotherapy Instrument", filed on October 16, 2024, the entire contents of which are incorporated herein by reference.

### TECHNICAL FIELD

The present application relates to the technical field of physiotherapy instruments, and particularly to a physiotherapy instrument.

### BACKGROUND

With the development of science and technology, the physiotherapy instrument, as a physiotherapy device, uses physiotherapy light to carry out corresponding physiotherapy on parts such as the hands, face or mouth. At this time, the physiotherapy light include red light, blue light or infrared light. In the related art, the existing physiotherapy instrument includes a plastic housing and a light source. The light source is provided inside the plastic housing and outputs light during operation. However, the light source will generate heat during operation, and the heat is accumulated in the internal space of the housing and cannot be dissipated outward, resulting in poor heat dissipation effect of the light source of the existing physiotherapy instrument.

### SUMMARY

The purpose of the present application is to provide a physiotherapy instrument. The light source is provided inside the housing, and the light source outputs light and can generate heat during operation. The heat-conducting base is provided on the housing and supports the light source. The heat-conducting base is located between the housing and the light source. The heat-conducting base exchanges heat with the light source, transmits the heat generated by the light source to the housing, and dissipates the heat through the housing. At this time, the heat-conducting base is added relative to the light source, it is convenient for the heat generated by the light source during operation to be conducted to the housing through the heat-conducting base, thus realizing the heat dissipation of the light source, making full use of the heat exchange of the heat-conducting base and the housing, the heat dissipation area of the light source is further increased, avoiding the situation that the light source cannot dissipate heat, and improving the heat dissipation effect of the light source of the physiotherapy instrument.

In order to achieve the above purpose, the present application provides the following technical solutions.

A physiotherapy instrument includes:

a housing;

a light source, provided inside the housing, wherein the light source outputs light and generates heat during operation; and

a heat-conducting base, provided on the housing and supporting the light source;

where the heat-conducting base is located between the housing and the light source, the heat-conducting base exchanges heat with the light source, transmits the heat generated by the light source to the housing, and dissipates the heat through the housing.

Compared with the related art, the beneficial effects of the present application are as follows:

The present application provides a physiotherapy instrument. The light source is provided inside the housing, and the light source outputs light and can generate heat during operation. The heat-conducting base is provided on the housing and supports the light source. The heat-conducting base is located between the housing and the light source. The heat-conducting base exchanges heat with the light source, transmits the heat generated by the light source to the housing, and dissipates the heat through the housing. At this time, the heat-conducting base is added relative to the light source, it is convenient for the heat generated by the light source during operation to be conducted to the housing through the heat-conducting base, thus realizing the heat dissipation of the light source, making full use of the heat exchange of the heat-conducting base and the housing, the heat dissipation area of the light source is further increased, avoiding the situation that the light source cannot dissipate heat, and improving the heat dissipation effect of the light source of the physiotherapy instrument.

### BRIEF DESCRIPTION OF THE DRAWINGS

In order to more clearly illustrate the technical solutions in the embodiments of the present application, the following will briefly introduce the drawings required for the description of the embodiments. Obviously, the drawings in the following description are only some embodiments of the present application. For those skilled in the art, without creative efforts, other drawings can be obtained based on these drawings.

In order to more comprehensively understand the present application and its beneficial effects, the following will be described in conjunction with the drawings. Herein, the same reference numerals in the following description represent the same parts.

FIG. 1 shows a schematic diagram of the physiotherapy instrument of the present application.

FIG. 2 shows a cross-sectional view of the physiotherapy instrument of the present application.

FIG. 3 shows a partially enlarged view of part A in FIG. 2.

FIG. 4 shows an exploded view of the physiotherapy instrument of the present application.

FIG. 5 shows a schematic diagram of the housing of the physiotherapy instrument of the present application.

FIG. 6 shows a schematic diagram of the connection of the light source, heat-conducting base and circuit board of the physiotherapy instrument of the present application.

FIG. 7 shows a schematic diagram of the heat-conducting base of the physiotherapy instrument of the present application.

FIG. 8 shows a schematic diagram of the pen tip of the physiotherapy instrument of the present application.

FIG. 9 shows a schematic diagram of the connection sleeve of the physiotherapy instrument of the present application.

FIG. 10 shows a schematic diagram of the connection of the lens and the light guide rod of the physiotherapy instrument of the present application.

FIG. 11 shows a schematic diagram of the connection of the light source and the reflector cup of the physiotherapy instrument of the present application.

FIG. 12 shows a cross-sectional view of the connection between the light source and the reflector cup of the physiotherapy instrument of the present application.

### DETAILED DESCRIPTION OF THE EMBODIMENTS

The following will clearly and completely describe the technical solutions in the embodiments of the present application in conjunction with the accompanying drawings of the embodiments of the present application. Obviously, the described embodiments are only a part of the embodiments of the present application, rather than all of them. Based on the embodiments in the present application, all other embodiments obtained by those skilled in the art without creative efforts shall fall within the protection scope of the present application.

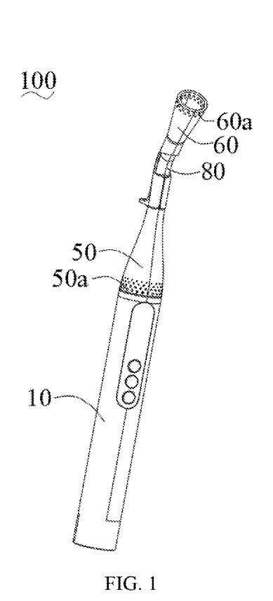

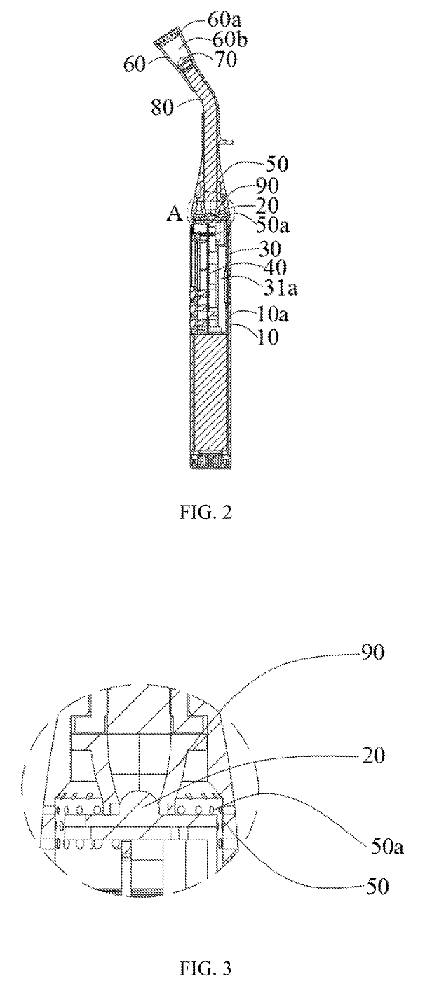

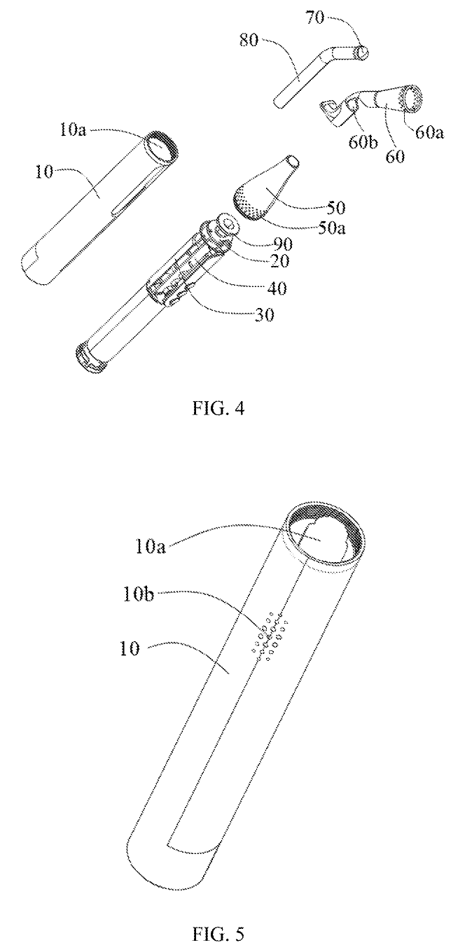

Please refer to FIGS. 1 to 4, the embodiment of the present application provides a physiotherapy instrument 100. As a physiotherapy device, the physiotherapy instrument 100 uses physiotherapy light to perform corresponding physiotherapy on parts such as the hands, face, or mouth. At this time, the physiotherapy lights include red light, blue light, or infrared light.

In the embodiment of the present application, the physiotherapy instrument 100 includes a housing 10, a light source 20, and a heat-conducting base 30. Both the light source 20 and the heat-conducting base 30 are provided inside the housing 10. The heat-conducting base 30 exchanges heat with the light source 20, transmits the heat generated by the light source 20 to the housing 10, and dissipates the heat through the housing 10. Since the heat-conducting base 30 is added relative to the light source 20, the heat generated by the light source 20 during operation can be conducted to the housing 10 through the heat-conducting base 30, thus realizing the heat dissipation of the light source 20, making full use of the heat exchange between the heat-conducting base 30 and the housing 10, the heat dissipation area of the light source 20 is further improved, avoiding that the light source 20 cannot dissipate heat, and thereby improving the heat dissipation effect of the light source 20 of the physiotherapy instrument 100.

Please refer to FIGS. 1 to 5, in the embodiment of the present application, the housing 10 serves as the supporting part 32 of the physiotherapy instrument 100, and the housing 10 is used to support the light source 20 and the heat-conducting base 30.

Please refer to FIGS. 1 to 4, in the embodiment of the present application, the light source 20 is provided inside the housing 10. The light source 20 outputs light and can generate heat during operation, so that the light output by the light source 20 can be conducted to the external environment.

Please refer to FIGS. 1 to 4 and 6, in the embodiment of the present application, the heat-conducting base 30 is provided inside the housing 10. The heat-conducting base 30 is provided on the housing 10 and supports the light source 20. The heat-conducting base 30 is located between the housing 10 and the light source 20. The heat-conducting base 30 exchanges heat with the light source 20, transmits the heat generated by the light source 20 to the housing 10, and dissipates the heat through the housing 10. At this time, since the heat-conducting base 30 is added relative to the light source 20, the heat generated by the light source 20 during operation can be conducted to the housing 10 through the heat-conducting base 30, thus realizing the heat dissipation of the light source 20, making full use of the heat exchange between the heat-conducting base 30 and the housing 10, the heat dissipation area of the light source 20 is further improved, avoiding that the light source 20 cannot dissipate heat, and thereby improving the heat dissipation effect of the light source 20 of the physiotherapy instrument 100.

Please refer to FIGS. 1 to 5, the housing 10 is provided with an inner cavity 10a. The inner cavity 10a serves as the internal space of the housing 10. Both the heat-conducting base 30 and the light source 20 are both located in the inner cavity 10a, so that both the heat-conducting base 30 and the light source 20 can be fixed at the inner side of the housing 10. Thus, the outer surface of the housing 10 can protect the heat-conducting base 30 and the light source 20, preventing the heat-conducting base 30 and the light source 20 from directly colliding with external objects. Optionally, the housing 10 is a heat-conducting pen body, so that the heat of the heat-conducting base 30 can be conducted to the heat-conducting pen body to achieve heat exchange between the heat-conducting base 30 and the heat-conducting pen body.

Please refer to FIGS. 1 to 4, 6 and 7. The heat-conducting base 30 includes a heat-conducting main body 31 and a supporting part 32. The light source is on the upper side of the supporting part 32. The supporting part 32 supports the light source 20, so that the light source 20 can be fixed on the supporting part 32, and thus the heat-conducting base 30 can support the light source 20 through the supporting part 32. The upper surface of the supporting part 32 is in contact with the lower surface of the light source 20, so that the supporting part 32 can exchange heat with the light source 20, and thus the heat generated by the light source 20 can be conducted from top to bottom to the supporting part 32. The heat-conducting main body 31 is located on the side of the supporting part 32 facing away from the light source 20. The heat-conducting main body 31 is connected to the supporting part 32, so that the heat-conducting main body 31 can be fixed on the supporting part 32, and thus the supporting part 32 can exchange heat with the heat-conducting main body 31, and then the heat of the supporting part 32 can be conducted to the heat-conducting main body 31.

Please refer to FIGS. 1 to 4, 6 and 7. The heat-conducting main body 31 is connected to the housing 10. The outer contour of the heat-conducting main body 31 is in contact with the inner contour of the housing 10 and exchanges heat with the housing 10, so that the heat of the heat-conducting main body 31 can be conducted to the housing 10. Thus, the heat generated by the light source 20 can be conducted to the housing 10 sequentially through the supporting part 32 and the heat-conducting main body 31, realizing the heat dissipation of the light source 20 and making full use of the heat exchange among the supporting part 32, the heat-conducting main body 31 and the housing 10.

Please refer to FIG. 7, the heat-conducting main body 31 and the supporting part 32 are integrated or detachable from each other, which improves the connection stability between the heat-conducting main body 31 and the supporting part 32 and ensures the connection strength between the heat-conducting main body 31 and the supporting part 32. Optionally, both the heat-conducting main body 31 and the supporting part 32 are heat-conducting members to achieve heat exchange between the heat-conducting main body 31 and the supporting part 32, so that the heat of the supporting part 32 can be conducted to the heat-conducting main body 31.

Please refer to FIGS. 1 to 4, the heat-conducting main body 31 is of an arc shape. The outer side wall of the heat-conducting main body 31 is adapted to the inner side wall of the housing 10 and is in contact with the inner side wall of the housing 10, so that the outer contour of the heat-conducting main body 31 is in contact with the inner side wall of the housing 10. Thus, the heat of the heat-conducting main body 31 can be conducted to the inner side wall of the housing 10 through the outer contour of the heat-conducting main body 31 to achieve heat exchange between the heat-conducting main body 31 and the housing 10.

Please refer to FIGS. 1 to 4 and 6, in the embodiment of the present application, the physiotherapy instrument 100 further includes a circuit board 40. The circuit board 40 is provided at the inner side of the heat-conducting main body 31. The circuit board 40 is provided on the heat-conducting main body 31, so that the outer side wall of the circuit board 40 is in contact with the inner side wall of the heat-conducting main body 31. The circuit board 40 exchanges heat with the heat-conducting main body 31, so that the heat of the circuit board 40 can be conducted to the heat-conducting main body 31. Thus, the heat generated by the circuit board 40 during operation can be conducted to the housing 10 through the heat-conducting main body 31, realizing the heat dissipation of the circuit board 40, making full use of the heat exchange between the heat-conducting main body 31 and the housing 10, the heat dissipation area of the circuit board 40 is further improved, avoiding that the circuit board 40 cannot dissipate heat, thereby improving the heat dissipation effect of the circuit board 40 of the physiotherapy instrument 100, so as to realize the simultaneous heat dissipation of the light source 20 and the circuit board 40.

Please refer to FIGS. 1 to 4, 6 and 7, the heat-conducting main body 31 is provided with an air duct 31a. The air duct 31a is used for air to pass through, and the air duct 31a is communicated with the inner cavity 10a. The housing 10 is provided with an air inlet hole 10b. The air inlet hole 10b is communicated with the external environment and the air duct 31a, and the air inlet hole 10b is docked with the air inlet of the air duct 31a, so that the air can flow into the air duct 31a sequentially through the air inlet hole 10b and the inner cavity 10a. The circuit board 40 is provided relative to the air duct 31a, so that the air in the air duct 31a can contact the circuit board 40, and thus the air in the air duct 31a can further dissipate the heat of the circuit board 40, improving the heat dissipation effect of the circuit board 40 of the physiotherapy instrument 100.

Please refer to FIGS. 1 to 4, 6 and 7, the air inlet of the air duct 31a is provided on the heat-conducting main body 31. There are multiple air inlets 10b. The multiple air inlets 10b are provided in a ring shape and are all communicated with the air inlet of the air duct 31a, so that the air in the external environment can flow into the air duct 31a through the multiple air inlets 10b. Since multiple air inlets 10b are provided, the flow efficiency of the air relative to the air duct 31a is increased, and the heat dissipation effect of the air on the circuit board 40 is improved.

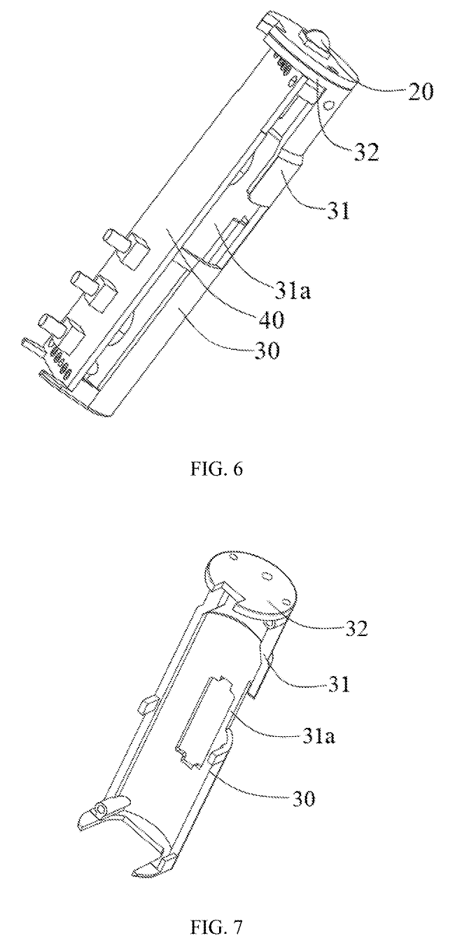

Please refer to FIG. 6, the circuit board 40 is positioned and connected to the heat-conducting main body 31, ensuring the position accuracy of the circuit board 40 relative to the heat-conducting main body 31, so that the heat-conducting main body 31 can limit the position of the circuit board 40, preventing the circuit board 40 from moving relative to the heat-conducting main body 31, and avoiding the bending and deformation of the circuit board 40.

Optionally, a fan is provided inside the housing 10. The fan is provided on one side of the heat-conducting base 30. The output end of the fan faces the heat-conducting base 30 and blows cold air to the heat-conducting base 30, so as to accelerate the heat dissipation of the heat-conducting base 30 and improve the heat dissipation effect of the heat-conducting base 30. At this time, the cold air output from the output end of the fan is discharged along the air duct 31a.

Please refer to FIGS. 1 to 4 and 8, in the embodiment of the present application, the physiotherapy instrument 100 further includes a pen tip 50. The pen tip 50 is provided on the front side of the housing 10 and is detachably connected to the housing 10, so that the pen tip 50 can be connected to or detached from the housing 10. When the pen tip 50 is connected to the housing 10, the pen tip 50 covers the light source 20, so that the pen tip 50 can condense the light output by the light source 20 and reduce the loss of the light output by the light source 20. When the pen tip 50 is detached from the housing 10, it is convenient to replace the pen tip 50, improving the convenience of replacing the pen tip 50. Optionally, the pen tip 50 is detachably connected to the housing 10 by the thread.

Please refer to FIGS. 1 to 4 and 8, the pen tip 50 is provided with an air outlet hole 50a. The air outlet hole 50a is communicated with the air duct 31a of the heat-conducting main body 31 and is docked with the air outlet of the air duct 31a, so that the air with the heat of the circuit board 40 can flow from the air outlet of the air duct 31a to the air outlet hole 50a sequentially and be discharged to the external environment through the air outlet hole 50a, realizing the discharge of the air with the heat of the circuit board 40. The air outlet hole 50a is close to the light source 20, so that the heat generated by the light source 20 during operation can be discharged to the external environment through the air outlet hole 50a, thus facilitating the further discharge of the heat generated by the light source 20 during operation and improving the heat dissipation effect of the light source 20.

Please refer to FIGS. 1 to 4 and 8, the pen tip 50 is of a conical shape. The pen tip 50 covers the light source 20. The outer diameter of the end of the pen tip 50 close to the housing 10 is larger than that of the end of the pen tip 50 away from the housing 10, so that the light output by the light source 20 can be converged through the pen tip 50 to achieve the condensing effect of the light output by the light source 20 and reduce the loss of light.

Please refer to FIGS. 1 to 4 and 8, the air outlet hole 50a is provided at one end of the pen tip 50 close to the housing 10. There are multiple air outlet holes 50a, and these multiple air outlet holes 50a are located around the light source 20, to facilitate the flow of the air with the heat of the circuit board 40 and the heat generated by the light source 20 during operation to the external environment through the multiple air outlet holes 50a. Since multiple air outlet holes 50a are provided, the discharge efficiency of the air with the heat of the circuit board 40 and the heat generated by the light source 20 during operation relative to the external environment is increased.

Please refer to FIGS. 1 to 4, 9 and 10, in the embodiment of the present application, the physiotherapy instrument 100 further includes a connection sleeve 60 and a lens 70. The connection sleeve 60 is located between the pen tip 50 and the lens 70. The connection sleeve 60 is docked with one end of the pen tip 50, so that the connection sleeve 60 can be fixed to the pen tip 50. The lens 70 is provided on the connection sleeve 60, so that the lens 70 can be fixed to the connection sleeve 60. Thus, the lens 70 can be connected to the pen tip 50 through the connection sleeve 60. The lens 70 is used to condense the light output by the light source 20, so that the lens 70 can condense the light output by the light source 20 again, further reducing the loss of light. Optionally, the lens 70 is integrally connected to the connection sleeve 60.

Please refer to FIGS. 1 to 4, 9 and 10, one end of the connection sleeve 60 away from the pen tip 50 is of a conical shape. The lens 70 is positioned and connected to the connection sleeve 60, ensuring the position accuracy of the lens 70 relative to the connection sleeve 60, so that the light output by the light source 20 can accurately irradiate the lens 70. The aperture of the end of the connection sleeve 60 away from the pen tip 50 is larger than the aperture of the light-emitting end of the lens 70, so that the end of the connection sleeve 60 away from the pen tip 50 can provide a certain supporting effect when it comes into contact with the user's skin, thus relieving the soreness in the hand during a long treatment time.

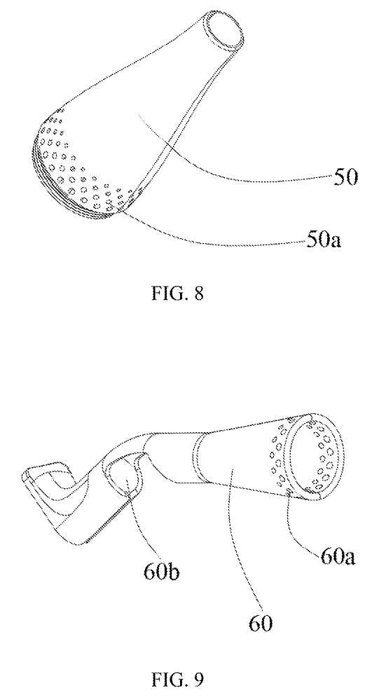

Please refer to FIG. 9, one end of the connection sleeve 60 away from the pen tip 50 is provided with a heat dissipation hole 60a. Specifically, the heat dissipation hole 60a is beneficial for heat dissipation during the treatment of the affected area, making the treatment more breathable and comfortable. Optionally, there are multiple heat dissipation holes 60, and these multiple heat dissipation holes 60 are provided in a ring along the connection sleeve 60. Since multiple heat dissipation holes 60 are provided, the heat dissipation effect of the connection sleeve 60 relative to the user is increased.

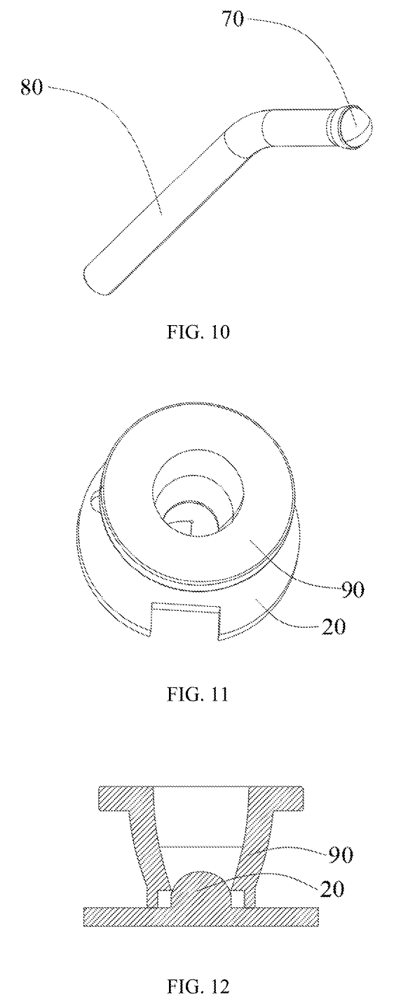

Please refer to FIGS. 9 to 10, the connection sleeve 60 is provided with a through hole 60b. The physiotherapy instrument 100 further includes a light guide rod 80. The outer contour of the light guide rod 80 is adapted to the inner contour of the through hole 60b. The light guide rod 80 passes through the through hole 60b and is docked with the light source 20 and the lens 70. The light guide rod 80 is used for conducting light, so that the light output by the light source 20 can be conducted to the lens 70 through the light guide rod 80, improving the conduction accuracy of the light output by the light source 20. Optionally, the lens 70 is connected to the connection sleeve 60 or the light guide rod 80; the lens is used for condensing the light output by the light source.

Please refer to FIGS. 9 to 10, the light guide rod 80 passes through the pen tip 50, the light guide rod 80 is docked with the light source 20, and the light guide rod 80 is used for transmitting the light output by the light source 20, so that the light can be provided relative to the part to be physiotherapized, realizing the physiotherapy of the part to be physiotherapized with the light.

In addition, the light guide rod 80 passes through the connection sleeve 60 and is positioned by the connection sleeve 60. There is an interference fit between the outer contour of the light guide rod 80 and the inner contour of the through hole 60b. The connection sleeve 60 is provided on the outer side of the light guide rod 80 and positions the light guide rod 80. At this time, the inner side wall of the connection sleeve 60 limits the position of the light guide rod 80, ensuring the position accuracy of the light guide rod 80 relative to the connection sleeve 60.

Please refer to FIGS. 11 to 12, in the embodiment of the present application, the physiotherapy instrument 100 further includes a reflector cup 90. The reflector cup 90 is provided inside the pen tip 50 and is sleeved on the light-emitting end of the light source 20. The inner side wall of the reflector cup 90 serves as a reflective surface, and this reflective surface is used for conducting the light output by the light source 20 to the light guide rod 80, so that the light output by the light source 20 can irradiate the light guide rod 80 under the action of the reflector cup 90, ensuring that the light output by the light source 20 propagates towards the light guide rod 80. Optionally, the reflective surface is a conical surface.

Compared with the related art, the beneficial effects of the present application are as follows:

the present application provides a physiotherapy instrument 100. The light source 20 is provided inside the housing 10. The light source 20 is provided inside the housing 10, and the light source 20 outputs light and can generate heat during operation. The heat-conducting base 30 is provided on the housing 10 and supports the light source 20. The heat-conducting base 30 is located between the housing 10 and the light source 20. The heat-conducting base 30 exchanges heat with the light source 20, transmits the heat generated by the light source 20 to the housing 10, and dissipates the heat through the housing 10. At this time, since the heat-conducting base 30 is added relative to the light source 20, the heat generated by the light source 20 during operation can be conducted to the housing 10 through the heat-conducting base 30, thus realizing the heat dissipation of the light source 20, making full use of the heat exchange between the heat-conducting base 30 and the housing 10, the heat dissipation area of the light source 20 is further increased, avoiding that the light source 20 cannot dissipate heat, thereby improving the heat dissipation effect of the light source 20 of the physiotherapy instrument 100.

At this time, the heat-conducting base 30 includes a heat-conducting main body 31 and a supporting part 32. The light source is on the upper side of the supporting part 32. The supporting part 32 supports the light source 20, so that the light source 20 can be fixed to the supporting part 32, and thus the heat-conducting base 30 can support the light source 20 through the supporting part 32. The upper surface of the supporting part 32 is in contact with the lower surface of the light source 20, so that the supporting part 32 can exchange heat with the light source 20, and thus the heat generated by the light source 20 can be conducted from top to bottom to the supporting part 32. The heat-conducting main body 31 is located on the side of the supporting part 32 facing away from the light source 20. The heat-conducting main body 31 is connected to the supporting part 32, so that the heat-conducting main body 31 can be fixed on the supporting part 32, and thus the supporting part 32 can exchange heat with the heat-conducting main body 31, and then the heat of the supporting part 32 can be conducted to the heat-conducting main body 31.

The heat-conducting main body 31 is connected to the housing 10. The outer contour of the heat-conducting main body 31 is in contact with the inner contour of the housing 10 and exchanges heat with the housing 10, so that the heat of the heat-conducting main body 31 can be conducted to the housing 10. Thus, the heat generated by the light source 20 can be conducted to the housing 10 sequentially through the supporting part 32 and the heat-conducting main body 31, realizing the heat dissipation of the light source 20 and making full use of the heat exchange among the supporting part 32, the heat-conducting main body 31 and the housing 10.

In addition, the heat-conducting main body 31 and the supporting part 32 are integrated or detachable from each other, which improves the connection stability between the heat-conducting main body 31 and the supporting part 32 and ensures the connection strength between the heat-conducting main body 31 and the supporting part 32. Optionally, both the heat-conducting main body 31 and the supporting part 32 are heat-conducting members to achieve heat exchange between the heat-conducting main body 31 and the supporting part 32, so that the heat of the supporting part 32 can be conducted to the heat-conducting main body 31.

Furthermore, the heat-conducting main body 31 is of an arc shape. The outer side wall of the heat-conducting main body 31 is adapted to the inner side wall of the housing 10 and is in contact with the inner side wall of the housing 10, so that the outer contour of the heat-conducting main body 31 is in contact with the inner side wall of the housing 10. Thus, the heat of the heat-conducting main body 31 can be conducted to the inner side wall of the housing 10 through the outer contour of the heat-conducting main body 31 to achieve heat exchange between the heat-conducting main body 31 and the housing 10.

The physiotherapy instrument 100 further includes a circuit board 40. The circuit board 40 is provided inside the heat-conducting main body 31. The circuit board 40 is provided on the heat-conducting main body 31, so that the outer side wall of the circuit board 40 is in contact with the inner side wall of the heat-conducting main body 31. The circuit board 40 exchanges heat with the heat-conducting main body 31, so that the heat of the circuit board 40 can be conducted to the heat-conducting main body 31. Thus, the heat generated by the circuit board 40 during operation can be conducted to the housing 10 through the heat-conducting main body 31, realizing the heat dissipation of the circuit board 40, making full use of the heat exchange between the heat-conducting main body 31 and the housing 10, the heat dissipation area of the circuit board 40 is further increased, avoiding that the circuit board 40 cannot dissipate heat, thereby improving the heat dissipation effect of the circuit board 40 of the physiotherapy instrument 100, so as to realize the simultaneous heat dissipation of the light source 20 and the circuit board 40.

The heat-conducting main body 31 is provided with an air duct 31a. The air duct 31a is used for air to pass through, and the air duct 31a is communicated with the inner cavity 10a. The housing 10 is provided with an air inlet hole 10b. The air inlet hole 10b is communicated with the external environment and the air duct 31a, and the air inlet hole 10b is docked with the air inlet of the air duct 31a, so that the air can flow from the air inlet hole 10b and the inner cavity 10a into the air duct 31a sequentially. The circuit board 40 is provided relative to the air duct 31a, so that the air in the air duct 31a can contact the circuit board 40, and thus the air in the air duct 31a can further dissipate the heat of the circuit board 40, improving the heat dissipation effect of the circuit board 40 of the physiotherapy instrument 100.

The air inlet of the air duct 31a is provided on the heat-conducting main body 31. There are multiple air inlet holes 10b. The multiple air inlet holes 10b are provided in a ring shape and are communicated with the air inlet of the air duct 31a, so that the air in the external environment can flow into the air duct 31a through the multiple air inlet holes 10b. Since multiple air inlet holes 10b are provided, the flow efficiency of the air relative to the air duct 31a is increased, and the heat dissipation effect of the air on the circuit board 40 is improved.

The circuit board 40 is positioned and connected to the heat-conducting main body 31, ensuring the position accuracy of the circuit board 40 relative to the heat-conducting main body 31, so that the heat-conducting main body 31 can limit the position of the circuit board 40, preventing the circuit board 40 from moving relative to the heat-conducting main body 31, and avoiding the bending and deformation of the circuit board 40.

In the above embodiments, the description of each embodiment has its own focus. For the parts not described in detail in a certain embodiment, reference may be made to the related descriptions of other embodiments. In the description of the present application, the terms "first" and "second" are only used for descriptive purposes and cannot be construed as indicating or implying relative importance or implicitly indicating the quantity of the indicated technical features. Thus, the features defined with "first" and "second" may explicitly or implicitly include one or more of these features.

Specific examples are applied in this text to illustrate the principle and implementation of the present application. The descriptions of the above embodiments are only used to help understand the method and its core idea of the present application. And for those skilled in the art, according to the idea of the present application, there will be changes in the specific implementation manners and application scopes. In summary, the content of this specification should not be construed as a limitation to the present application.

What is claimed is:

1. A physiotherapy instrument, comprising:
a housing;
a light source, provided inside the housing, wherein the light source outputs light and generates heat during operation; and
a heat-conducting base, provided on the housing and supporting the light source;
wherein the heat-conducting base is located between the housing and the light source, the heat-conducting base exchanges heat with the light source, transmits the heat generated by the light source to the housing, and dissipates the heat through the housing.

2. The physiotherapy instrument according to claim 1, wherein the housing is provided with an inner cavity, and the heat-conducting base and the light source are located in the inner cavity, and the housing is a heat-conducting pen body.

3. The physiotherapy instrument according to claim 2, wherein the heat-conducting base comprises a heat-conducting main body and a supporting part;
the supporting part supports the light source and is in contact with a side wall of the light source;
the heat-conducting main body is connected to the supporting part, and the heat-conducting main body is connected to the housing and exchanges heat with the housing; and
the heat-conducting main body and the supporting part are heat-conducting members.

4. The physiotherapy instrument according to claim 3, wherein the heat-conducting main body is of an arc shape, the heat-conducting main body is adapted to an inner side wall of the housing and is in contact with the inner side wall of the housing.

5. The physiotherapy instrument according to claim 3, wherein the physiotherapy instrument further comprises a circuit board, the circuit board is provided on the heat-conducting main body, and the circuit board exchanges heat with the heat-conducting main body.

6. The physiotherapy instrument according to claim 3, wherein the heat-conducting main body is provided with an air duct for air to pass through, and the air duct is communicated with the inner cavity; and
the housing is provided with an air inlet hole, the air inlet hole is communicated with an external environment and the air duct, and the air inlet hole is docked with an air inlet of the air duct.

7. The physiotherapy instrument according to claim 1, further comprising a pen tip; wherein the pen tip is provided on one side of the housing and is detachably connected to the housing; and
the pen tip is provided with an air outlet hole, the air outlet hole is communicated with the air duct of the heat-conducting main body and is docked with the air outlet of the air duct.

8. The physiotherapy instrument according to claim 7, wherein the physiotherapy instrument further comprises a light guide rod, the light guide rod passes through and is provided at the pen tip, the light guide rod is docked with the light source for transmitting the light output by the light source.

9. The physiotherapy instrument according to claim 8, wherein the physiotherapy instrument further comprises a connection sleeve and a lens;
the connection sleeve is provided on an outer side of the light guide rod and positions the light guide rod;
the lens is connected to the connection sleeve or the light guide rod; and
the lens is configured for condensing the light output by the light source.

10. The physiotherapy instrument according to claim 8, wherein the light guide rod passes through the connection sleeve and is positioned by the connection sleeve;
the connection sleeve is located on an outer peripheral side of the light guide rod;
the physiotherapy instrument further comprises a reflector cup, the reflector cup is provided inside the pen tip and is sleeved on a light-emitting end of the light source; and
an inner side wall of the reflector cup serves as a reflective surface for conducting the light output by the light source to the light guide rod.

11. The physiotherapy instrument according to claim 6, wherein the air inlet of the air duct is provided on the heat-conducting main body; and
multiple air inlet holes are of a ring shape and are communicated with the air inlet of the air duct.

12. The physiotherapy instrument according to claim 6, wherein a fan is provided inside the housing, an output end of the fan faces the heat-conducting base and blows cold air to the heat-conducting base.

13. The physiotherapy instrument according to claim 7, wherein the pen tip is of a conical shape, the pen tip covers the light source, and an outer diameter of an end of the pen tip close to the housing is larger than an outer diameter of an end of the pen tip away from the housing, to make the light output by the light source be converged through the pen tip.

14. The physiotherapy instrument according to claim 9, wherein one end of the connection sleeve away from the pen tip is of a conical shape, the lens is positioned and connected to the connection sleeve, and one end of the connection sleeve away from the pen tip is provided with a heat dissipation hole.

15. A physiotherapy instrument, comprising:
a housing;
a light source, provided inside the housing, wherein the light source outputs light and generates heat during operation;
a heat-conducting base, located between the housing and the light source;
wherein the heat-conducting base exchanges heat with the light source, transmits the heat generated by the light source to the housing, and dissipates the heat through the housing; and
the heat-conducting base comprises a heat-conducting main body and a supporting part; the heat-conducting main body and the supporting part are integrated or detachable from each other.

16. The physiotherapy instrument according to claim 15, wherein the heat-conducting main body and the supporting part are heat-conducting components.

17. The physiotherapy instrument according to claim 15, wherein the heat-conducting main body is of an arc shape, the heat-conducting main body is adapted to an inner side wall of the housing and is in contact with the inner side wall of the housing.

18. The physiotherapy instrument according to claim 15, wherein the heat-conducting main body is provided with an air duct for air to pass through, and the air duct is communicated with the inner cavity of the housing; and
the housing is provided with an air inlet hole, the air inlet hole is communicated with an external environment and the air duct, and the air inlet hole is docked with an air inlet of the air duct.

19. A physiotherapy instrument, comprising:
a housing, being a heat-conducting pen body;
a light source, provided inside the housing, wherein the light source outputs light and generates heat during operation; and
a heat-conducting base, located between the housing and the light source;
wherein the heat-conducting base exchanges heat with the light source, transmits the heat generated by the light source to the housing, and dissipates the heat through the housing.

20. The physiotherapy instrument according to claim 19, wherein a fan is provided inside the housing, the fan is provided on one side of the heat-conducting base, an output end of the fan faces the heat-conducting base and blows cold air to the heat-conducting base, the cold air output from the output end of the fan is discharged along an air duct;
the physiotherapy instrument further comprises a pen tip; the pen tip is provided on one side of the housing and is detachably connected to the housing; and
the pen tip is provided with an air outlet hole, the air outlet hole is communicated with the air duct of the heat-conducting main body and is docked with an air outlet of the air duct.
